(19) United States
(12) Patent Application Publication
(10) Pub. No.: US 2009/0123935 A1
(43) Pub. Date: May 14, 2009

(54) Measurement of a population of nucleic acids, in particular by real time PCR

(76) Inventors: Virginie Jahan, Rueil Malmaison (FR); Karine Kaminski, Juvignac (FR)

Correspondence Address: NIXON & VANDERHYE, PC, 901 NORTH GLEBE ROAD, 11TH FLOOR, ARLINGTON, VA, 22203, US

(21) Appl. No.: 12/289,405

(22) Filed: Oct. 27, 2008

Related U.S. Application Data
(60) Provisional application No. 60/992,876, filed on Dec. 6, 2007.

(30) Foreign Application Priority Data
Oct. 25, 2007 (FR) 0707508

(51) Int. Cl. C12Q 1/68; C12M 1/34

(52) U.S. Cl. 435/6; 435/287.2

(57) ABSTRACT

The invention relates to a method for measuring the amount of nucleic acids of a target sequence in a sample of interest. The method comprises several steps, and in particular subjecting the sample of interest to an amplification treatment, in the presence of at least one nucleic acids label specific for said target sequence; measuring a physical quantity representative of the evolution of the label; calculating a parameter F₀ representative of the physical quantity of the nucleic acids label before any amplification cycle; estimating the initial population size of nucleic acids of the target sequence using a conversion law, comprising contextual parameters, and applied to said parameter F₀. The contextual parameters are pre-recorded reference parameters which are substantially independent of at least a part of the experimental conditions. The invention is also directed towards an apparatus comprising units organized for implementing a method for measuring the amount of nucleic acids of a target sequence in a sample of interest.

The present application claims benefit of U.S. Provisional Application No. 60/992,876, filed Dec. 6, 2007, and FR 0707508, filed Oct. 25,2007, the entire contents of each of which is hereby incorporated by reference.

The present invention relates to a method for measuring an initial amount of nucleic acids contained in a sample of interest and subjected to an amplification. The invention finds in particular an advantageous application in the therapeutic monitoring of infectious diseases, but also in the genetics field and the field of cancer research and treatment, and, more generally, regardless of the field of application, for instance the veterinary field, food testing or the plant field.

The field concerned is that of gene amplification techniques for determining an initial amount of a nucleic acid sequence of interest present in a sample, and in particular amplification techniques for real time quantification, such as the real time PCR (polymerase chain reaction) technique, for example.

The present invention finds an advantageous but non-limiting application in the determination of an initial amount of nucleic acids in a sample subjected to a real time PCR reaction in order to evaluate the number of copies of nucleic acids of infectious agents (viruses, bacteria, yeasts, etc.) present in a biological sample (serum, plasma, blood, expectorations, etc.). This technique can work on DNA or RNA. Reference will here be made, generally and unless otherwise mentioned, to DNA, without however being restricted to this nucleic acid.

PCR involves temperature variations which allow dissociation of the DNA into two strands (“denaturation”) and then hybridization of a primer on each strand of the DNA thus denatured. Each primer is therefore specific for and complementary to one of the two DNA strands. The action of a DNA polymerase subsequently leads to the synthesis of new complementary strands of each strand initially used as template. This forms a series of “denaturation/hybridization/strand synthesis” steps, which is repeated cyclically.

Real time PCR is a technique allowing the simultaneous amplification and detection of one or more different sequences of target nucleic acids. The detection can be carried out over the course of time and can be quantitative.

The quantitative real time PCR technique involves one or more DNA labels, for example fluorescent labels, which allow the quantitation of the nucleic sequence (or of the nucleic sequences) to be detected. This technique generally uses the comparison of the sample to be analysed with a standard, or with a range of standards (dilution series of a standard). A standard is a sample containing a known amount of nucleic acids. It undergoes, in principle, the same treatments as the sample to be analysed. For the sample of interest as for the standard, the fluorescence evolves proportionally to the amount of nucleic acids present in the sample before and during the reaction. This will be described in detail below in the subsequent description.

Moreover, quantitative real time PCR encompasses several phases, which have different reaction dynamic, as will be seen later. Consequently, several points of measurement are generally used in order to monitor the reaction dynamics.

All this means that the real time PCR techniques for the quantitation of a sample of interest are laborious to implement, thus limiting the generalisation of their use, everywhere they are clearly of interest.

The present invention improves the situation.

To this effect the invention introduces a method for measuring the amount of nucleic acids of a target sequence in a sample of interest, comprising the steps of:
a. subjecting the sample of interest to an amplification treatment, with a batch of reagents comprising at least one nucleic acid(s) label specific for said target sequence, said amplification treatment comprising successive amplification cycles (i; from 1 to n),
b. measuring a physical quantity (Fᵢ) representative of the evolution of the label for at least a part of the amplification cycles,
c. expressing a parameter (F₀) representative of the physical quantity of the nucleic acid(s) before any amplification cycle, using the measurements carried out in step b.,
d. estimating the initial population size (N₀^{sam}) of nucleic acid(s) of the target sequence using a conversion law, comprising contextual parameters, and applied to said parameter (F₀) expressed in step c.

In step d., said contextual parameters are pre-recorded reference parameters, these parameters being substantially independent of at least a part of the experimental conditions.

The reference parameters mentioned above are determined in advance based on the behaviour of at least one reference sample of the same biological format as the sample of interest and of known initial population size N₀^{ref} of nucleic acid(s).

According to one embodiment, the conversion law in step d. can be applied directly, without the need for correction by the execution in parallel of steps a., b. and c. for at least one calibrator.

This embodiment therefore simplifies the implementation of the method for the end-user by reducing the time consuming and also allows to a significant reduction in reagent costs, in particular in the public health domain. Furthermore, by virtue of the pre-recorded reference parameters, the handling of elements which conventionally accompany an amplification reaction is avoided. In particular, the use of standard samples is avoided, thereby making it possible to increase the number of wells available for testing the samples to be analysed (place gain).

Another embodiment comprises step d. a correction of the estimated initial population size of nucleic acids (N₀^{sam}). This correction is based on carrying out in advance steps a., b. and c. for at least one calibrator (EQC) of known initial population size of nucleic acid(s) and of known parameter (F₀^{EQC}) representative of the physical quantity of the nucleic acid(s) label before any amplification cycle.

This correction of the estimated initial population size (N₀^{sam}) can be carried out by means of the following steps:
establishing a corrective law between:
   said known parameter (F₀^{EQC}) representative of the physical quantity of the nucleic acid(s) label before any amplification cycle, and
   the effectively expressed parameter (F₀^{EQCmeasured}) representative of the physical quantity of the nucleic acid(s) label before any amplification cycle, of the calibrator,
applying said corrective law to said conversion law comprising the contextual parameters of step d., in order to estimate said initial size (N₀^{sam}) of the population of nucleic acid(s) of the target sequence, present in the sample of interest.

For the abovementioned correction, the calibrator (EQC) of known initial population size is based on a biological substance able to form a positive control for the sample of interest.

In this embodiment, the correction does not therefore generate additional reaction tubes, which is advantageous compared with the known quantitation techniques of the prior art.

In step c. of the method, the expression of the parameter (F₀) involves at least:
one parameter relating to the switch between a first phase of constant amplification yield and a second phase of non-constant amplification yield,
one parameter relating to the constant amplification yield during said first phase, and
one parameter relating to the non-constant amplification yield during said second phase.

The nucleic acids label can be a fluorescent label.

The sample of interest can be a biological specimen that may comprise a pathogenic agent.

The amplification reaction used may be a real time polymerase chain reaction (PCR).

The reference parameters preferably have the advantage of being independent of at least one of the elements of the group of following experimental conditions: apparatus used, type of apparatus used, operator, period of validity of the batch of reagents, method of extraction of the nucleic acids of the target sequence.

According to another embodiment and in the case of a “multiplex PCR” amplification treatment, the sample of interest may comprise several distinct target sequences to be quantified.

The present invention is also directed towards an apparatus for measuring the amount of nucleic acids of at least one target sequence in a sample of interest, comprising:
a. a carrier element-for carrying at least one sample comprising a batch of reagents and the target sequence with at least one nucleic acid(s) label specific for said target sequence,
b. an amplification unit for subjecting the sample of interest to an amplification treatment comprising successive amplification cycles (i; from 1 to n),
c. a measuring unit for measuring a physical quantity (Fᵢ) representative of the evolution of said label for at least a part of the amplification cycles,
d. a processing unit comprising a memory and organized so as:
   i. to express a parameter (F₀) representative of the physical quantity of the nucleic acid(s) label prior to any of the amplification cycles, on the basis of measurements of physical quantity,
   ii. to estimate the initial population size (N₀^{sam}) of nucleic acid(s) of a target sequence present in the sample of interest using a conversion law, on the basis of contextual parameters,
e. a controller organized so that, when a sample of interest is received on the carrier element, it applies the amplification unit and the measuring unit to said sample received, and calls up the processing unit with the measurements obtained by said measuring unit.

In this apparatus, the contextual parameters estimated with the processing unit are reference parameters which are substantially independent of at least a part of the experimental conditions and are pre-recorded in the processing unit.

The present invention is also directed towards a computer program product comprising instructions for implementing the abovementioned method. This program is intended to be stored in a memory of the processing unit in the apparatus described above. In addition, the invention is directed towards a data storage support comprising instructions for implementing the method described above.

Other advantages and characteristics of the invention will become more apparent while reading the detailed description hereinafter and on the attached figures on which:

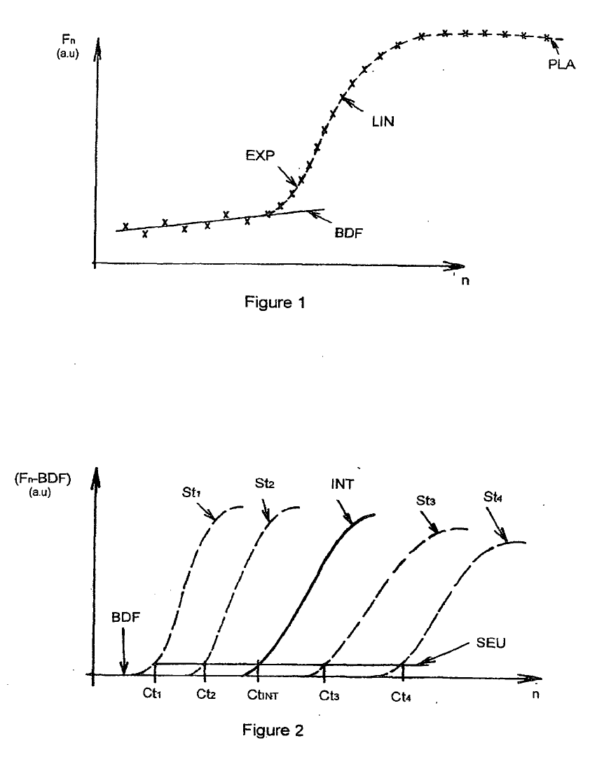

FIG. 1 shows a curve of evolution of a polymerase chain reaction (PCR),

FIG. 2 shows measurements of fluorescence that are emitted as a function of the number of PCR cycles,

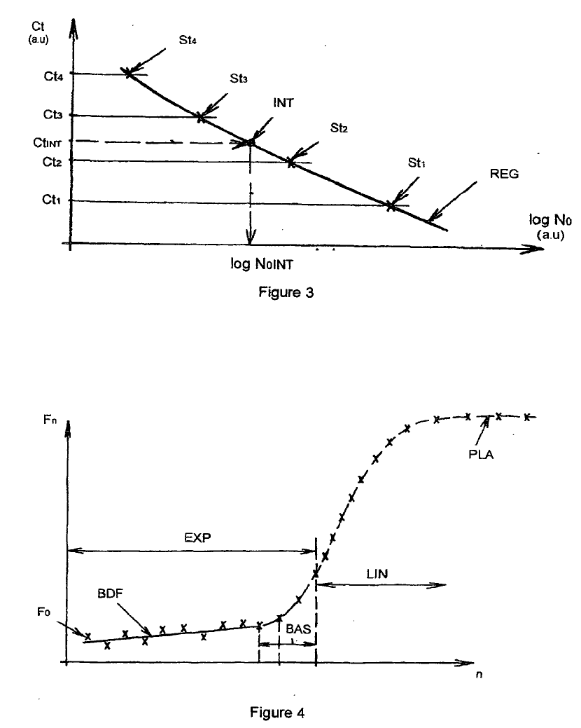

FIG. 3 relates to the prior art for the determination of the initial population of nucleic acids of a target sequence in a sample of interest and shows a regression curve established using the Ct method,

FIG. 4 relates to the determination of a physical quantity (F₀) representative of the initial amount of a target sequence present in a sample of interest (prior art),

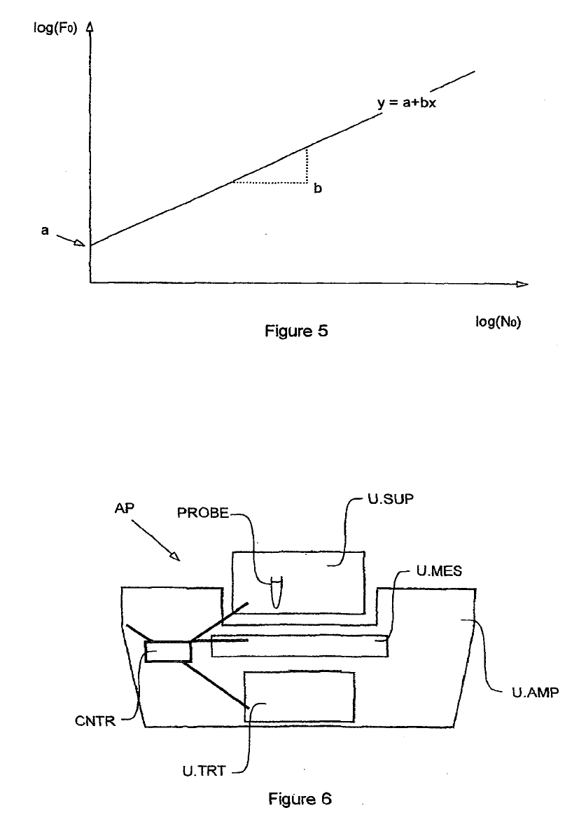

FIG. 5 shows a standard curve (straight) established from reference samples of the same biological format as the sample to be analysed,

FIG. 6 represents, schematically and functionally, an apparatus for quantifying an initial population of a sample of interest,

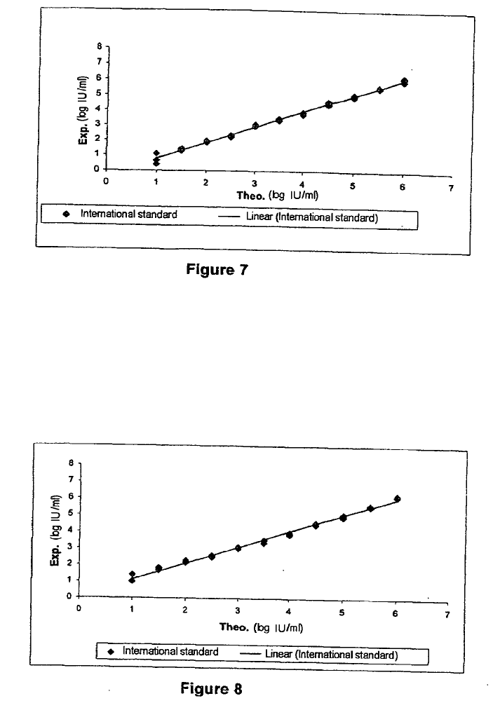

FIG. 7 shows a curve representative of the accuracy of a quantitation method according to the prior art, and

FIG. 8 shows a curve representative of the accuracy of an example of implementation of the invention.

The drawings and the description hereinafter mostly contain elements of definite nature. They may therefore not only serve to explain the present invention more clearly, but also to contribute to its definition, where appropriate.

The techniques for quantifying the population of nucleic acids present in a sample of interest will now be returned to in greater detail. The term “sample of interest” defines substantially a sample to be analysed. This sample of interest may therefore be any biological sample (body fluid, in particular) that may comprise at least one nucleic acid target sequence to be quantified. The term “population (or population of interest) size of nucleic acids” defines, in general, the amount of one or more target sequences present in a sample of interest.

As a general rule, the population of nucleic acids present in the sample of interest is too low to allow them to be simply quantified by direct measurement. The sample of interest is therefore subjected to an amplification treatment in view to amplify the target sequence(s) to be quantified.

The amplification treatment consists, firstly, in extracting the target sequence(s) from the sample of interest by conventional extraction methods known in the art, and, secondly, in subjecting the target sequence(s) to a succession of applications of an amplification reaction. It will therefore be understood that the sample of interest evolves as the amplification treatment advances. Starting from a crude biological specimen (blood or serum, for example), the sample of interest undergoes several reaction phases (extraction, purification, washing, etc.) involving various chemical and/or biological reagents. The biochemical composition of the sample of interest is not therefore fixed as the amplification treatment advances. Those skilled in the art will be able to distinguish the biochemical composition in view of the description and in particular will be able to differentiate between:
the “crude” sample of interest, which defines the crude biological specimen such as, in particular, blood, serum or a skin specimen,
the “extracted” sample of interest, which defines the sample after extraction and purification of the nucleic acids comprising one or more target sequences to be quantified,
the “reactional” sample of interest, which defines the sample conditioned for the purpose of undergoing a series of applications of an amplification reaction; the sample comprises one or more target sequences to be quantified and also a batch of reagents necessary for applying said succession of applications of an amplification reaction to the sample (described in detail below).

The succession of applications of an amplification reaction can, for example, be the “real time PCR” technique. However, the invention is not necessarily limited to this technique and could apply to any amplification treatment (and could in particular apply to LCR, ligase chain reaction).

The real time PCR technique in particular finds its application in the case of diagnosis and therapeutic monitoring for evaluating the number of copies of pathogenic agents (for example, of the hepatitis B virus: HBV, of the hepatitis C virus: HCV or else of the human immunodeficiency virus: HIV) in a specimen of body fluids (for example, serum, plasma or blood) of an individual.

In PCR, the quantitation is carried out at the end of the reaction, generally on an agarose gel. It is based on the number of nucleic acids (population size) present at the end of an amplification reaction of a target sequence, and on the comparison with one or more nucleic acid sequences present in a standard sample or several standard samples (e.g. dilution series of a standard), the initial concentration(s) of which is (are) known.

When a single standard sample is used, it is generally amplified in parallel to the target sequence. In general, these methods are quite imprecise and not very sensitive, hence an unsatisfactory quantitation. The development of real time PCR has brought about greater precision and sensitivity.

When a real time PCR is carried out, the quantitation is carried out over the course of the amplification reaction. Labels, for example fluorescent labels, specific for the target sequence to be amplified are used. Various systems involving fluorescent labels currently exist. Among these systems, mention should be made of those described in documents U.S. Pat. No. 5,210,015, U.S. Pat. No. 5,487,972, U.S. Pat. No. 5,723,591, U.S. Pat. No. 5,118,801, U.S. Pat. No. 5,925,517 and U.S. Pat. No. 6,150,097. The fluorescence emitted during the reaction, and more specifically emitted after each amplification cycle, evolves proportionally to the amount of PCR products, i.e. with the amount of nucleic acids, more specifically, with the amount of target sequence(s). Consequently, the entire reaction kinetics can theoretically be; measured by this method. In practice, measurement is possible only from the moment when the measurement—the fluorescence—can be distinguished from a background noise.

Very briefly, the term “amplification cycle” or “PCR cycle” is intended to mean a phase during which the sample of interest undergoes temperature variations in the presence of a certain number of reagents (dNTPs, polymerase, primers, etc.). The temperature variations make it possible, firstly, to denature the double-stranded DNA by cleaving the secondary structures such that the two strands are dissociated. Subsequently, the temperature variations will allow the specific DNA primers, present in the reaction medium, to hybridize to the target sequence, and will allow activation of the polymerases which will carry out the synthesis of new DNA strands (such a strand is referred to as an “amplicon” and is complementary to the target sequence). In real time PCR, it is the collection of target sequences which is detected at each new cycle by the fluorescent labels.

The schematic appearance of a real time PCR amplification curve is represented in FIG. 1, with PCR cycle indices along the x-axis and, along the y-axis, amounts of fluorescence emitted (in arbitrary units [a.u]). At each PCR cycle, the fluorescence is measured.

The amount of fluorescence Fₙ evolves as a function of the number n of PCR cycles already carried out. The following are successively distinguished in this evolution:
a first part BDF, where the fluorescence measurements are substantially indistinguishable from the background noise of the apparatus for measuring fluorescence,
a second part EXP, where the amounts of fluorescence measured increase substantially exponentially,
a third part LIN, where the increase in the amounts of fluorescence measured is substantially reduced and behaves, overall, in a substantially linear manner, and
a fourth part PLA, where the fluorescence measurements reach a plateau phase (saturation effect).

During the first and second parts defined above, the reaction kinetics correspond to an exponential law. Over the course of these cycles, the population of interest (amount of target sequence) increases constantly in an exponential manner. A subsequently stationary exponential phase of the amplification reaction is thus defined.

The cycles which follow during the third and fourth phases no longer obey exponential kinetics. This is because the growth of the population of interest starts to compete with damping phenomena, in particular the limitation of dNTP, fluorescent labels and degradation of the enzymatic activity of the polymerase. In fact, a plateau phase will be reached. This is a non-stationary phase of the amplification reaction.

Various techniques have been described for estimating the unknown initial amount of nucleic acids of a target sequence in a sample of interest. The document “Mathematics of quantitative kinetic PCR and the application of standard curves”, by R. G. Rutledge and C. Côté, in the scientific journal Nucleic Acids Research, 2003, vol. 31, No. 16, describes a method which consists in using several samples of known initial amounts of nucleic acids, referred to as “standards”, for determining, by interpolation, the initial amount of nucleic acids present in the sample of interest. FIG. 2 relates to this prior art.

As mentioned above, by relating the amount of fluorescence measured Fₙ to the number n of PCR cycles, a first part, BDF, is distinguished in which the fluorescence measurements merge with a background noise from the fluorescence-measuring apparatus. As the PCR reaction progresses, the fluorescence reaches a fluorescence threshold which can be distinguished from the background noise. This threshold is generally preset arbitrarily by the operator and is substantially above the background noise.

It is noted that, the greater the initial amount of nucleic acids of a target sequence in a sample of interest, the earlier the fluorescence can be distinguished from the background noise. This is directly linked to the fact that, the greater the initial amount of nucleic acids of a target sequence, the earlier an amount of PCR product is reached that is large enough to allow a signal discernible from the background noise.

FIG. 2 shows several standards of initial amounts of nucleic acids of a sequence that are known. It is understood that the initial population in the standard St₁ is higher than that of the standard St₂, which is higher that that of the standard St₃, and so on. In view of the above, the fluorescence distinction cycle therefore occurs earlier for the standard St₁, than for the standard St₂, which also occurs before the distinction cycle for the standard St₃, and so on.

Consequently, by setting a fluorescence threshold SEU distinct from the background noise BDF and common to all the standards, it is noted that the number Ct₁ of PCR cycles required for the standard St₁ to reach this preset threshold is, consequently, lower than the number Ct₂ of PCR cycles required for the standard St₂ to reach this threshold, which is lower than the number Ct₃ of PCR cycles required for the standard St₃ to reach this threshold, etc.

This observation has been taken advantage of in the prior art for establishing a dependency as represented in FIG. 3 between the number of cycles Ct₁, Ct₂, Ct₃, Ct₄, for several standards of known initial population size N₀^{St1}, N₀^{St2}, N₀^{St3}, N₀^{St4}. This dependency can be visualized by means of a curve, referred to as a regression curve REG. Thus, by plotting the cycles Ct₁, Ct₂, etc. along the y-axis, and the logarithm of the initial population size N₀^{St1}, N₀^{st2}, etc. along the x-axis, the regression curve REG is obtained. In the present case, it is more specifically a regression line. This REG line will subsequently be used for determining the initial population size of nucleic acids of a target sequence in a sample of interest. Thus, FIG. 2 shows a sample of interest of unknown initial population size INT, of which a cycle Ct_{INT} will be distinguished. By a projection of the cycle Ct_{INT} onto the REG regression curve/line, it will subsequently be possible to determine the initial size N₀^{INT} of the population of the sample of interest (see FIG. 3).

Although widely used, this method nevertheless has a certain number of drawbacks, of which the three principal ones are:
that of being entirely based on the judgment of the operator for the determination of the value for the fluorescence threshold SEU that can be distinguished from the background noise. Now, it is difficult to accurately distinguish such a threshold due to the fact that the fluorescence measurements in this region (typically at the BDF/EXP transition) are very close to the background noise BDF,
that of requiring the use of several standard samples for which the initial population size of nucleic acids of one or more sequences are known, which makes this method time-consuming and expensive,
that of accepting that the yield from amplification of the nucleic acid population is identical for all the standard samples and for the sample of interest; when the sample of interest contains PCR inhibitors, the estimation of the initial population size will be false since it will be too low.

In order to address the abovementioned drawbacks and to provide other advantages, and in particular the use of a single standard, document EP1700145 introduced a method implemented by computer-based means for absolute and/or relative quantitation of an initial population of nucleic acids in a sample of interest. In this process, the sample is subjected to a succession of applications of an amplification reaction for a population of interest (one or more target sequences). The amplification may be a PCR or any other amplification technique, provided that it is possible to follow the variation in yield from the reaction corresponding to this amplification. In fact, the deduction of the initial amount of nucleic acids in the sample uses the fact that, in the context of reactions to amplify an amount of nucleic acids, the yield often shifts from a constant efficiency to a non-constant efficiency. To define this shift BAS, experimental measurements representative of a current size of the population are taken during the course of the amplification, so as to finally deduce the initial population size in the sample of interest.

In real time PCR, the shift BAS mentioned above corresponds substantially to the shift in amplification yield observed between the constant phase and the non-constant phase, both defined above. This shift is typically here between the exponential phase (part EXP) and the linear phase (part LIN). It is by means of this shift that the inventors of the present application have been able to introduce (EP1700145) the method of quantitation for a nucleic acid population present in a sample of interest mentioned above. In order to be able to determine with precision the shift BAS, it is necessary to exploit virtually all the points of measurement of the amplification curve. For this reason, real time PCR is particularly suitable for the implementation of this method.

FIG. 4 relates to the determination of the initial amount of nucleic acids of a target sequence according to document EP1700145, which also involves a parameter F₀, which corresponds to the value of the fluorescence for the population of the sample of interest under consideration, before any amplification. This parameter F₀, which cannot be measured since it is below the background noise BDF, is theoretical but substantially representative of the initial population size. The determination of the parameter F₀ is what gives the method greater precision compared with the prior art. This is because the quantitation of the initial population size of a nucleic acid population in the prior art is carried out by setting a fluorescence threshold in the exponential phase EXP, typically at the end of the part BDF (Ct method). As detailed above, it is difficult to establish the threshold with precision. For this reason, the method described in EP1700145 exploits almost all points of the amplification curve, resulting in greater precision than the known methods.

To this effect, it is firstly necessary to form the following hypotheses:
during the first amplification cycles at the time of the exponential part EXP, the reaction yield is substantially constant, and
the reaction yield decreases starting from a certain number of amplification cycles; this decrease takes place during the third and fourth parts, i.e. the linear part LIN and the plateau part PLA respectively.

It should be noted that the background noise is an experimental problem since, in theory, the exponential phase begins from the first cycle onwards. The decrease in yield can be explained in various ways, as mentioned above, and in particular degradation of the reaction reagents (DNA polymerases, dNTPs, primers, etc.) or alternatively inhibition by products formed during the reaction.

In any event, the determination of the parameter F₀ requires, in general, a transition from a constant yield, corresponding to a situation of increase per amplification, to a non-constant yield. It is understood that, for a non-constant yield, this may be a decreasing or increasing yield. For the implementation of the invention, it is sufficient to detect a shift in yield from a constant phase to a non-constant phase.

For a yield Eₙ from the reaction of the amplification at cycle n and constant up to cycle k, it may be written:

Eₖ=Eₖ₋₁=Eₖ₋₂=. . . E₀, where E₀ is the value of the yield of the constant phase.

The size of the population of interest (one or more target sequences) at an amplification cycle is given by the relationship: Nₙ₊₁=Nₙ+Eₙ.Nₙ, where:
Nₙ is the size of the population of interest after an amplification of index n in an amplification series,
N₊₁ is the size of the population of interest after an amplification following an amplification of index n during the amplification series, and
Eₙ is the yield from the amplification reaction of index n.

By recurrence, this equation may be written: Nₙ₊₁=(1+E₀)ⁿ⁺¹.N₀, where N₀ is the initial size of the population of interest.

Once the constant phase has been passed, i.e. when the index n+1 goes beyond the region of shift, the relationship becomes: Nₙ₊₁=N₀×(1+E₀)^{Ceep}×function Cₑₑₚ, n+1), where:
the index Cₑₑₚ itself represents the index of shift, per se, between the exponential phase and the linear phase; it will therefore be understood that (Cₑₑₚ−1) is the last index of the amplification reaction during which the yield is still constant;
the term function (Cₑₑₚ, n+1) is a specific function which characterizes the non-constant phase of the yield and which depends at least on the shift index Cₑₑₚ and on the current index of the amplification n+1. It will be noted that the shift index Cₑₑₚ and the initial size N₀ of the population of interest are linked by this relationship.

By the subtraction of the background noise, and also a certain number of subsequent compensations and statistical relationships, it will be possible to determine the theoretical initial fluorescence F₀ before the first amplification cycle. Document EP1700145, incorporated herein by way of reference and to which the reader may make reference, describes the determination of the parameter F₀ in greater detail.

In the present description, the parameter F₀ refers to a fluorescent signal. However, the invention is in no way limited to such a feature. In fact, any other physical quantity that can be measured before the first amplification cycle and representative of the initial population size of nucleic acids of a target sequence may be used for the implementation of the invention. However, in the interests of simplicity and comprehension, the subsequent description will be limited to the embodiment using a fluorescent signal. For the purpose of the present invention, this fluorescent signal is emitted after hybridization of the fluorescent label to the nucleic acid target sequence (“molecular beacon” method). Consequently, only the target sequence(s) and the amplicons, in association with the fluorescent label, emit said fluorescent signal.

Using, therefore, as a basis, the principle that the fluorescence emitted is proportional to the amount of nucleic acids present in the sample analysed, before and during the amplification reaction, it results therefrom that:

F₀=k×N₀P, with k being a real constant and p being close to 1.

The equation of the REG regression curve represented in FIG. 5 and describing a relationship of proportionality between an initial concentration of a target sequence of interest and the virtual parameter F₀, is of the type:

log (F₀)=a+b log (N₀) (F1)

This equation is of the form y=a+bx, and describes a straight line for which the constants a and b, i.e. precisely the intercept a and the slope b, may be defined. However, in other cases, the equation of the REG regression curve does not follow a straight-line equation, but may, for example, be of the polynomial or sigmoidal type, or the like. In this case, the number of constants may increase or decrease according to the complexity of the equation of the REG curve. In the interests of simplicity, the embodiment described here is limited to the case of a regression curve in the form of a straight line and thus to the determination of the intercept a and the slope b.

The REG curve/straight line is pre-established using a series of standard sample of the same biological format as the sample of interest (“reference samples” —detailed below) and more precisely using a set of standard sample dilution series, hereinafter referred as range. The term “biological format” will be intended to mean, in the rest of the present description, the combination:
biological nature of the target sequence present in a sample, and
conditioning of the sample.

The biological nature is substantially defined by the target sequence to be quantified in a sample (sample of interest or standard sample/reference sample). It may therefore be DNA or RNA (genomic, plasmid, etc.) and, more specifically, the identity of a nucleic acid sequence (complete sequence of HBV, fragment of HBV, complete sequence of HIV, fragment of HIV, etc.).

The “conditioning of a sample” defines:
at least the reagent batch essential for carrying out the amplification reaction and accompanying this sample in the reaction medium, and
the treatment to which the sample had been subjected from the extraction to the determination of the parameter F₀.

The appearance of the curve REG and, consequently, of the constants a and b depends substantially on the biological format of the sample to be analysed.

The applicant has discovered, not unsurprisingly, that regression curves established using the parameter F₀ show a low intervariability of the values of a and b when the biological format of various samples is identical. This stability is related to the parameter F₀.

In the subsequent description, reference will be made to the abovementioned standard samples as reference samples. A reference sample is therefore: any sample of the same biological format as the sample of interest and for which the initial concentration of nucleic acids of a target sequence is known. For the purpose of the invention, a reference sample therefore comprises one or more sequences of the same biological nature as the target sequence(s) to be quantified. Furthermore, the reference samples comprise a reagent batch identical to that of the sample of interest, and have undergone substantially the same treatment from extraction to determination of the parameter F₀ (same conditioning—see above).

The purpose of the distinction which is made between the reference samples (used for the invention) and the standard samples (as used for the Ct method) is in particular to specify the fact that the operator manipulating the reference samples is different from the operator manipulating the standard samples. In fact, in the subsequent description, it will be clearly apparent that any reference sample manipulation is carried out in the factory/industrially and not by the end-user of the invention.

More generally, the applicant has discovered that, for the quantitation of a target sequence of a given biological format, it is sufficient to establish a standard curve REG involving a relationship between a physical quantity representative of the initial size of a population and the known concentration of at least two reference samples. In the embodiment described, the standard curve corresponds to a linear law. By means of a conversion law, the slope and the intercept (reference parameters) of this curve make it possible, in a subsequent or joint step, to determine the initial population size of nucleic acid of the target sequence(s).

A determination of the values of a and b and also the determination of an initial concentration of a target sequence are given below in an implementation example. Prior to this, a certain number of terms used in the rest of the description are defined.

In the embodiment described hereinafter, the sample of interest comprises nucleic acids extracted and isolated by extraction/purification from a sample of human serum or plasma. The nucleic acid(s) to be quantified (also referred to as target sequence(s) or alternatively target sequence(s) of interest) are, for example, viral genomic DNA. The target sequence(s) is (are) amplified and quantified by real time PCR. In the examples described, it will more specifically be genomic DNA of the hepatitis B virus (HBV), the size of which is approximately 3.2 kb. Of course, the invention is in no way limited to this example.

The term “internal control” (IC) is intended to mean, in the implementation example described, an amplified fragment of genomic DNA of a plant organism of known concentration, the sequence of which is heterologous to that of the target sequence present in the sample to be analysed. The internal control is added to the samples of interest and to the negative control (defined below) from the beginning of the extraction onwards. It is co-amplified with the target sequence. The internal control makes it possible, firstly, to be sure that the extraction has been carried out satisfactorily and, secondly, to control the lack of inhibitor in the samples of interest. To this effect, it should be noted that the size, the GC (guanine/cytosine) percentage and the efficiency of the amplification reaction are substantially identical to those of the target sequence. In the embodiment described, the IC is diluted in a TE buffer solution (10 mM Tris HCl, 0.5 mM EDTA) pH 8.3 and Proclin™ 300. The implementation of the invention does not require the presence of the IC to be taken into account, due to the fact that it has no effect on the amplification of the target sequence.

In parallel to the amplification of the target sequence of interest, a positive control (PC) can be used to verify that the reagents specific for the amplification of said target sequence to be quantified are functioning correctly. In general, the reagents in the reaction mixture containing the positive control are substantially identical to those of the sample of interest. In particular, the primers are specific both for the target sequence and for the positive control.

In the context of a specific embodiment, the positive control may also be used as sole calibrator (details given below) if it proves necessary for the quantitation of the sample of interest. This calibrator is referred to as EQC (External Quantitative Calibrator).

In the implementation example described here, the positive control or EQC consists of an amplified fragment of viral genomic DNA of the hepatitis B virus (HBV), of which both the initial amount and the confidence interval for the parameter F₀ are known. The size and also the sequence of the EQC are identical to those of the target sequence of interest. It is amplified with an efficiency substantially identical to that of the target sequence in the sample of interest. It is an external calibrator which is not co-amplified with the samples of interest. The calibrator is amplified in parallel. This means that the calibrator is either amplified At the same time or in a distinct step with respect to the sample of interest. The positive control or EQC therefore, firstly, makes it possible to validate the PCR reagents specific for the sample of interest and, secondly, can serve, according to a second embodiment of the invention, as calibrator for the method for measuring the amount of nucleic acids (details given below). In the embodiment described, the positive control/EQC is diluted in TE buffer (10 mM Tris HCl, 0.5 mM EDTA) pH 8.3. According to other embodiments which are not described here, the positive control/EQC may be diluted in plasma, serum, or the like.

Of course, the reaction mixture may be accompanied by any control which makes it possible to qualify the amplification reaction. Such a control may, for example, be a negative control (NC). In the implementation example described, the negative control is a sample of defibrinated human plasma that is negative for HBV genomic DNA and contains Proclin™ 300. In other words, it is negative for the target sequence of interest of the implementation example. This negative control is added to each series of extraction of the nucleic acids from the samples of interest and is subjected to the same amplification treatment as this sample, i.e. the extraction/purification process and the real time PCR. In general, a negative control makes it possible to be sure of the lack of any contamination in the samples of interest. Such contaminations may occur during the extraction phase or during the preparation of the real time PCR. Furthermore, together with the internal control, the negative control makes it possible to control the validity of the IC-specific PCR reagents.

As indicated above, the embodiment described here is based on the principle that the fluorescence of a sample of interest, and more specifically of a reactional sample of interest, is proportional to the amount of one or more target sequences present in this sample, this being the case both before and during the amplification reaction. This observation will emerge clearly in the light of the non-limiting implementation example described below.

### EXAMPLES

The extraction/purification and also the amplification reaction applied to the reference samples and samples of interest (real time PCR) were carried out according to a conventional method of the art and in a substantially identical manner. The experimental protocol used is described hereinafter.

### I. Experimental Protocol

### I-1. Reference Samples

Accurun 325 positive control DNA of the hepatitis B virus—series 700 (BBI Diagnostic—Seracare, reference A325-5723).

### I-2. Nucleic Acid Extraction and Purification

The Accurun sample is diluted in human plasma negative for the presence of HBV in order to constitute a range of reference samples of various concentrations of target sequence nucleic acids.

Each dilution point is extracted in the presence of IC (the concentration of which is 300 copies/PCR).

The HBV viral DNA is extracted using the QIAamp DSP Virus Kit (QIAGEN, reference 60704) according to the protocol of the kit.

### I-3. Amplification Reaction: Real Time PCR

Oliqonucleotide sequences used

Primer and probe sequences

| | | |
|---|---|---|
| HBV | Primer 1 | 5′ - GCT GAA TCC CGC GGA CGA - 3′ |
| system | Primer 2 | 5′ - GTG CAG AGG TGA AGC GAA |
| | | GTG - 3′ |
| | Probe 1 | 5′ FAM - CGG CAG GAG TCC GCG TAA |
| | | AGA GAG GTG TGC CG - Dabcyl 3′ |
| IC | Primer 3 | 5′ - GAG CCG CAG ATC CGA GCT A - 3′ |
| system | Primer 4 | 5′ - GGA GTG GAA CAT AGC CGT |
| | | GGTC - 3′ |
| | Probe 2 | 5′ Atto647N - TGC TGC GTC CTC CGC |
| | | CGC CAC CGC TTG GGC AGC A - |
| | | Dabcyl 3′ |

reactional mixture:
   HBV/Fam Dabcyl—IC/Atto647N Dabcyl multiplex
0.6 μM of the HBV molecular beacon probe (Eurogentec), 0.6 μM of the 2 HBV primers, 0.2 μM of the IC molecular beacon probe (Eurogentec), 0.3 μM of the 2 IC primers, 2.5 U of HotStarTaq polymerase (QIAGEN, reference 203205), 6 mM MgCl₂, 200 μM d(ACGU)TP, 100 μM dTTP, 0.25 U UDG, 0.3% PVP, 5% glycerol.
   Thermoprofile on the amplification apparatus (Chromo4):

$$30 min at 42 ° C . 15 min at 95 ° C . \\ \begin{matrix}15 sec at 95 ° C . \\ 30 sec at 55 ° C . \\ 30 sec at 72 ° C .\end{matrix} \} 50 X$$

$$20 ° C .$$

### I-4. Interpretation of the Results

For each sample (reference samples or samples of interest), the parameter F₀ is determined according to the method of document EP 1 700 145.

### II. Results

II-1. Establishment of a Standard Curve from a Range of Reference Samples using the Parameter F₀.

FIG. 5 shows a standard curve (straight), established on a logarithmic scale, which reveals the physical parameter F₀ (fluorescence before any amplification cycle) as a function of the initial concentration of a target sequence of the reference samples. The reference points used to establish this curve are those of a range of reference samples of known initial concentrations expressed in IU/PCR (international units/reaction volume), and the parameter F₀ of which was calculated according to the method of patent EP1700145 (see experimental protocol above).

For the purposes of accuracy, F₀ is determined for 2 HBV replicates per concentration point (columns “Replicate 1” and “Replicate 2” of Table I).

Table I below reveals these values.

TABLE I

| | HBV F₀ | | | |
|---|---|---|---|---|
| N₀ in IU HBV | Value of | Value of | | Standard |
| DNA/PCR | Replicate 1 | Replicate 2 | Mean | deviation |
| 1.10⁵ | 4.15E−09 | 3.44E−09 | 3.79E−09 | 4.99E−10 |
| 1.10⁴ | 1.65E−10 | 1.53E−10 | 1.59E−10 | 8.58E−12 |
| 1000 | 1.09E−11 | 9.56E−12 | 1.02E−11 | 9.46E−13 |
| 100 | 7.08E−13 | 5.47E−13 | 6.28E−13 | 1.14E−13 |
| 10 | 6.23E−14 | 6.47E−14 | 6.35E−14 | 1.71E−15 |

It will therefore be learned from this that, firstly, prior to determining the initial amount of nucleic acids of the target sequence present in the sample of interest, it is necessary to establish a standard curve based on at least two reference samples of biological format identical to said sample of interest and of known initial nucleic acid concentrations. Based on the standard curve, and more specifically standard curve in the present example, the intercept and the slope (reference parameters a and b) can be determined by conventional means known to those skilled in the art.

As described in the experimental protocol, these measurements were accompanied by an internal control IC in order to validate the reagents and to control the extraction step and also the lack of inhibitor.

### II-2. Determination of the Reference Parameters a and b

For a precise determination of a and of b, i.e., respectively, of the intercept and of the slope, a multitude of ranges of reference samples were analysed. Of course, the invention can be carried out without this refining. However, in the implementation example present here, 22 ranges of reference samples are used to establish:
a median value of a, and
a median value of b.

TABLE II

| Reference sample | | |
|---|---|---|
| range | Intercept in IU/ml (a) | Slope (b) |
| Range 1 | −14.989 | 1.112 |
| Range 2 | −15.445 | 1.220 |
| Range 3 | −15.234 | 1.188 |
| Range 4 | −15.159 | 1.169 |
| Range 5 | −15.258 | 1.204 |
| Range 6 | −15.324 | 1.196 |
| Range 7 | −15.582 | 1.245 |
| Range 8 | −15.836 | 1.295 |
| Range 9 | −15.449 | 1.222 |
| Range 10 | −15.538 | 1.229 |
| Range 11 | −15.452 | 1.226 |
| Range 12 | −14.803 | 1.052 |
| Range 13 | −15.075 | 1.139 |
| Range 14 | −14.907 | 1.103 |
| Range 15 | −15.031 | 1.151 |
| Range 16 | −15.151 | 1.185 |
| Range 17 | −15.113 | 1.135 |
| Range 18 | −14.889 | 1.091 |
| Range 19 | −14.742 | 1.065 |
| Range 20 | −15.185 | 1.136 |
| Range 21 | −15.337 | 1.181 |
| Range 22 | −14.717 | 1.029 |

It emerges from Table II that the variability of the values a and b (intervariability) between the various ranges of reference samples is low, due in particular to the reliability and the robustness of the parameter F₀.

The values of a and of b were calculated, respectively, from the median of both the slope and the intercept (see Table III). It should be recalled here that, for each reference sample, the biological format is identical to that of the sample of interest. For a sample of interest with a given biological format, a and b are therefore set for a given batch of reagents. It should be noted that, for establishing a and b, any statistical value representative of all the measurements, such as, for example, the mean (see Table III) can be used.

TABLE III

| Intercept (a) | | | Slope (b) | | |
|---|---|---|---|---|---|
| | | Standard | | | Standard |
| Mean | Median | deviation | Mean | Median | deviation |
| −15.192 | −15.172 | 0.29 | 1.163 | 1.175 | 0.07 |

The values given in Tables II and III show the low variability of a and of b. The quantitation of the initial population N₀ in a sample of interest can then be carried out on the basis of formula F2, deduced from formula F1:

$${N}_{0} = {10}^{\frac{log ( {F}_{0} ) - a}{b}}$$

Starting, therefore, from a source sample similar to a sample of interest but of known initial concentration (hereinafter referred to as test sample), the accuracy of the quantitation test according to the invention can be verified.

### II-3. Example 1—Quantitation of Three Distinct Test Samples

In this example, the Accurun 325 positive control hepatitis B virus DNA—Series 700 sample (BBI Diagnostic, reference A325-5723) was diluted to three different concentrations (test samples A, B and C) in human plasma negative for the presence of HBV. These three test samples have a known initial concentration of target sequence (see Table IV).

The nucleic acids of the test samples were extracted and purified in the presence of IC, using the QIAamp DSP Virus Kit (QIAGEN, reference 60704) according to the protocol of the kit. The test samples were subjected to an amplification treatment with the same reagent batch as that used to calculate the parameters a and b in the previous example.

### II-3.a) Measurement of the F₀ Values of the Three Test Samples

TABLE IV

| | HBV F₀ | | | |
|---|---|---|---|---|
| | Value of | Value of | | |
| | Replicate | Replicate | | Standard |
| Sample | 1 | 2 | Mean | deviation |
| NC 0 IU/ml | 0 | 0 | 0 | 0 |
| Test A 19.1 IU/ml | 4.15E−14 | 2.00E−14 | 3.08E−14 | 1.52E−14 |
| Test B 1.91.10³ IU/ml | 3.28E−12 | 4.05E−12 | 3.67E−12 | 5.45E−13 |
| Test C 1.91.10⁵ IU/ml | 8.85E−10 | 1.12E−09 | 1.00E−09 | 1.67E−10 |

### II.3.b) Quantitation

The parameters a and b for the HBV strain of the above implementation example have the values −15.172 and 1.175, respectively (median value, see Table III). The initial concentrations are determined/verified using the reference parameters (a and b) in formula F2. The results are given in Table V and show that the quantitation with these values is very satisfactory (quantitation error less than a factor of two).

TABLE V

| | Log | | Mean Log | | | Mean | Standard |
|---|---|---|---|---|---|---|---|
| | N₀ in | | N₀ in | N₀ in | | N₀ in | Deviation |
| Sample | IU/ml | | IU/ml | IU/ml | | IU/ml | IU/ml |
| NC 0 IU/ml | — | — | — | — | — | — | — |
| Test A | 1.524 | 1.254 | 1.389 | 33 | 18 | 26 | 11 |
| 19.1 IU/ml | | | | | | | |
| Test B | 3.138 | 3.216 | 3.177 | 1375 | 1645 | 1510 | 191 |
| 1.91.10³ IU/ml | | | | | | | |
| Test C | 5.206 | 5.294 | 5.250 | 1.61E+05 | 1.97E+05 | 1.79E+05 | 2.53E+04 |
| 1.91.10⁵ IU/ml | | | | | | | |

II-4. Example 2—Comparative Example of Quantitation of an HBV International Standard (International Standard: WHO international standard for HBV DNA nucleic acid amplification techniques 97/746)

This is a comparative example between the determination of the initial population size of a target sequence present in a sample of interest according to, firstly, the Ct method and, secondly, the method according to the invention. The parameters a and b determined in the previous example were used for the determination of the initial population. The example is based on the use of the international standard referred to as WHO (WHO international standard for HBV DNA nucleic acid amplification techniques 97/746).

Two series of half-log dilutions of the WHO international standard were carried out in human plasma negative for the presence of HBV. Each dilution point was extracted/purified in the presence of IC, using the QIAamp DSP Virus Kit (QIAGEN, reference 60704) according to the protocol of the kit. Each extract was amplified by real time PCR in duplicate.

In a manner similar to Example 1, the values F₀ were determined for each sample of interest. The target sequence contained in each sample is subsequently quantified using formula F2. Table VI gives the results of the quantitations expressed as a logarithmic scale.

TABLE VI

| | Ct method | | | Method according to the invention | | |
|---|---|---|---|---|---|---|
| | International Standard IU/ml [log] | | | International Standard IU/ml [log] | | |
| | Theoretical | Experimental | | Theoretical | Experimental | |
| | values | values | Mean | values | Values | Mean |
| 1st | 6 | 6.184 | 6.168 | 6 | 6.239 | 6.174 |
| series | 6 | 6.268 | | 6 | 6.239 | |
| 2nd | 6 | 6.203 | | 6 | 6.19 | |
| series | 6 | 6.016 | | 6 | 6.158 | |
| 1st | 5.5 | 5.606 | 5.593 | 5.5 | 5.618 | 5.593 |
| series | 5.5 | 5.597 | | 5.5 | 5.604 | |
| 2nd | 5.5 | 5.543 | | 5.5 | 5.596 | |
| series | 5.5 | 5.63 | | 5.5 | 5.564 | |
| 1st | 5 | 4.991 | 5.023 | 5 | 5.044 | 5.001 |
| series | 5 | 5.107 | | 5 | 5.063 | |
| 2nd | 5 | 5.065 | | 5 | 4.974 | |
| series | 5 | 4.931 | | 5 | 4.924 | |
| 1st | 4.5 | 4.465 | 4.525 | 4.5 | 4.456 | 4.512 |
| series | 4.5 | 4.576 | | 4.5 | 4.569 | |
| 2nd | 4.5 | 4.43 | | 4.5 | 4.454 | |
| series | 4.5 | 4.631 | | 4.5 | 4.569 | |
| 1st | 4 | 3.877 | 3.86 | 4 | 3.932 | 3.908 |
| series | 4 | 3.919 | | 4 | 3.982 | |
| 2nd | 4 | 3.749 | | 4 | 3.869 | |
| series | 4 | 3.896 | | 4 | 3.851 | |
| 1st | 3.5 | 3.474 | 3.403 | 3.5 | 3.521 | 3.43 |
| series | 3.5 | 3.388 | | 3.5 | 3.49 | |
| 2nd | 3.5 | 3.343 | | 3.5 | 3.353 | |
| series | 3.5 | 3.409 | | 3.5 | 3.354 | |
| 1st | 3 | 3.07 | 2.989 | 3 | 3.099 | 3.052 |
| series | 3 | 2.94 | | 3 | 3.094 | |
| 2nd | 3 | 2.959 | | 3 | 3.002 | |
| series | 3 | 2.987 | | 3 | 3.012 | |
| 1st | 2.5 | 2.327 | 2.284 | 2.5 | 2.581 | 2.511 |
| series | 2.5 | 2.213 | | 2.5 | 2.475 | |
| 2nd | 2.5 | 2.252 | | 2.5 | 2.47 | |
| series | 2.5 | 2.341 | | 2.5 | 2.517 | |
| 1st | 2 | 1.98 | 1.91 | 2 | 2.263 | 2.174 |
| series | 2 | 1.868 | | 2 | 2.132 | |
| 2nd | 2 | 1.854 | | 2 | 2.122 | |
| series | 2 | 1.938 | | 2 | 2.178 | |
| 1st | 1.5 | 1.417 | 1.379 | 1.5 | 1.793 | 1.767 |
| series | 1.5 | 1.417 | | 1.5 | 1.775 | |
| 2nd | 1.5 | 1.292 | | 1.5 | 1.681 | |
| series | 1.5 | 1.39 | | 1.5 | 1.819 | |
| 1st | 1 | 1.089 | 0.714 | 1 | 1.396 | 1.128 |
| series | 1 | 0.413 | | 1 | 0.997 | |
| 2nd | 1 | 0.639 | | 1 | 0.992 | |
| series | 1 | | | 1 | | |
| 1st | 0.5 | −0.199 | −0.199 | 0.5 | 0.727 | 0.727 |
| series | 0.5 | | | 0.5 | | |
| 2nd | 0.5 | | | 0.5 | | |
| series | 0.5 | | | 0.5 | | |

FIGS. 7 and 8 show the accuracy, respectively, of the Ct method and of the method according to the invention with the theoretical values along the x-axis (in log IU/ml) and the experimental values along the y-axis (in log IU/ml)—The equations for the curve (straight) of FIGS. 7 and 8 are, respectively:

y=−0.2966+1.0682×; for the Ct method, and

y=0.1305+0.983×; for the method according to the invention.

The quantitation according to the invention is equivalent in terms of accuracy to the Ct method, and has all the advantages listed in the present description, with in particular the simplicity of quantitation due to the parameter F₀.

### II-4. Example 3—Quantitation of Clinical Samples

25 clinical samples originating from patients infected with HBV, quantified beforehand using a commercial hepatitis B virus quantitation kit (kit using the Ct method), were tested according to the quantitation method of the invention. The extraction, the purification and the amplification treatment are those of the experimental protocol described above.

Each target sequence in each of the clinical samples was amplified with the same batch of reagents as the one used to determine the values of a and b in the previous examples.

In a manner similar to Example 1, the values of F₀ were determined for each sample of interest. The initial population size in the samples was subsequently quantified using formula F2. Table VII gives the results of the quantitations.

Clinical samples

| | Quantitation Roche | | Quantitation according to the | | |
|---|---|---|---|---|---|
| | Cobas TaqMan | | invention | | |
| Sample | Log N₀ in | N₀ in | | Log N₀ in | N₀ in |
| No. | IU/ml | IU/ml | F₀ | IU/ml | IU/ml |
| 1 | 1.362 | 23 | 5.10E−14 | 1.600 | 40 |
| 2 | 3.856 | 7176 | 1.41E−11 | 3.676 | 4744 |
| 3 | 3.268 | 1855 | 3.20E−12 | 3.129 | 1345 |
| 4 | 3.403 | 2530 | 6.05E−12 | 3.364 | 2314 |
| 5 | 2.312 | 205 | 3.20E−13 | 2.277 | 189 |
| 6 | 2.509 | 323 | 4.88E−13 | 2.434 | 271 |
| 7 | 2.938 | 866 | 1.31E−12 | 2.798 | 628 |
| 8 | 2.303 | 201 | 2.99E−13 | 2.253 | 179 |
| 9 | 2.620 | 417 | 6.52E−13 | 2.541 | 347 |
| 10 | 3.561 | 3637 | 1.40E−11 | 3.674 | 4726 |
| 11 | 2.958 | 908 | 3.46E−12 | 3.157 | 1436 |
| 12 | 2.149 | 141 | 2.51E−13 | 2.188 | 154 |
| 13 | 4.111 | 12915 | 3.98E−11 | 4.060 | 11479 |
| 14 | 3.208 | 1613 | 1.85E−12 | 2.927 | 845 |
| 15 | 3.410 | 2572 | 5.75E−12 | 3.346 | 2216 |
| 16 | 4.098 | 12528 | 3.16E−11 | 3.975 | 9432 |
| 17 | 3.548 | 3533 | 5.03E−12 | 3.296 | 1976 |
| 18 | 2.923 | 838 | 2.57E−12 | 3.048 | 1117 |
| 19 | 3.719 | 5231 | 8.26E−12 | 3.479 | 3016 |
| 20 | 2.286 | 193 | 1.43E−13 | 1.981 | 96 |
| 21 | 2.959 | 910 | 2.27E−12 | 3.001 | 1003 |
| 22 | 5.282 | 191392 | 2.30E−09 | 5.559 | 362265 |
| 23 | 2.773 | 593 | 1.43E−12 | 2.832 | 679 |
| 24 | 2.624 | 421 | 7.22E−13 | 2.578 | 379 |
| 25 | 2.407 | 255 | 4.66E−13 | 2.417 | 261 |

The results obtained according to the method of the invention on the clinical samples are equivalent to the quantitations obtained with a commercial reference kit, while at the same time exhibiting the advantages resulting from the method according to the invention. The previous example underlines the fact that the invention meets the regulatory requirements and the strict accuracy of quantitation required for medical tests, such as the quantitation of viral nucleic acids in a patient's sample.

The above examples illustrate the simplicity with which a final operator can carry out a quantitation of a population of nucleic acids by means of the method according to the invention. In fact, at least two steps, i.e.: establishing a standard curve and determining the constant parameters of the equation of the standard curve, are carried out in the factory/industrially and pre-recorded in a PCR apparatus according to the invention. The final operator will thus benefit from simplicity of use for carrying out the quantitation of one or more target sequences present in a sample of interest. Principally and by virtue of the pre-recorded parameters, the operator will bypass the two steps mentioned above. This results in time being gained compared with the methods of the prior art. Furthermore, the examples clearly show the robustness of the method. This robustness is due to the use of F₀.

In practice, the operator will merely have to subject the sample of interest to the amplification treatment, i.e. extraction/purification of the nucleic acids of the crude sample of interest, in order to obtain an extracted sample of interest, and then addition of a batch of reagents to the latter in order to obtain a reactional sample of interest, and, finally, to apply the reactional sample of interest to a carrier unit of an amplification apparatus. The amplification apparatus will determine the value of F₀ (according to the method described in EP1700145) and, by means of this value F₀ and together with the pre-recorded parameters (standard curve equation constants), will determine by itself the initial amount of nucleic acids of one or more target sequences present in the sample of interest. All this means that the invention is of great economical advantage.

Of course, the values of a and of b vary according to the biological nature of the target sequence. As detailed above, these values also vary according to the batch of reagents applied to the sample of interest in order to carry out the amplification reaction. However, for a given batch of reagents, the values a and b are stable over time throughout the duration of validity of the batch, i.e. until its expiry date. This is synonymous with a high degree of reliability for the final operator.

More generally, the values of a and of b are independent of:
the apparatus used for the amplification treatment (in particular of a same commercial model),
the type of apparatus used (it is therefore possible to use any type of commercial model, in particular the apparatus known as “Chromo 4” from the company Bio-Rad or the apparatus known as “LightCycler” from the company Roche),
the handling site (transportability of the apparatus),
the operator carrying out the technical manipulations,
the validity of the batch of reagents,
individual components used in the batch of reagents (it is therefore possible to use any type of polymerase, of dNTP, etc.),
the method of extraction of the nucleic acids of the target sequence.

The use of the relationship between F₀ and N₀ seems to be responsible for the low variability and the stability of the values a and b.

By virtue of these advantages and the accuracy of the quantitation, the invention corresponds to the regulatory requirements demanded for medical tests, such as the quantitation of viral nucleic acids in a sample of interest from a patient.

According to this first embodiment of the invention and contrary to the prior art, it will therefore no longer be necessary for the operator to use a range of standards in order to determine the initial population of the sample of interest to be quantified. In fact, the initial population of a sample is determined by virtue of the predetermined and pre-recorded reference parameters, in particular a and b in the implementation examples, for a given biological format.

According to another embodiment, a single calibrator or EQC (external calibrator), which may be the positive control, can be used to perform a correction of the pre-recorded reference parameters, when the latter show a variability in precision of the determination of the initial population.

The correction uses the parameter F₀. More specifically, the set F₀ (hereinafter: F₀^{EQC}) of the EQC is determined in a step prior to the step for estimation of the initial size of the population of interest. This determination is therefore carried out before the application of said conversion law.

A simple comparison is then carried out between the value F₀ of the EQC measured in the apparatus (hereinafter: F₀^{EQCmeasured) and the known value F}₀^{EQC} in a confidence interval determined in the factory/industrially. A corrective proportionality relationship may then be established in order to compensate for the difference in values. The contextual parameters of step d. of the method according to the invention may therefore comprise correction parameters.

In embodiments as a variant, the correction may be based on other coefficients, with non-linear proportionality relationships.

More generally, it will therefore be sufficient to determine a reference physical quantity using a calibrator, and to calibrate the apparatus on this quantity.

The method described above is preferably carried out with a single apparatus equipped with distinct or combined units capable of carrying out the steps of said method and in particular capable of carrying out the amplification of one or more target sequences present in the sample of interest as described above.

Reference will be made to FIG. 6, which shows, schematically and functionally, an apparatus AP for quantifying an initial population of a sample of interest. This apparatus AP comprises a carrier unit U.SUP capable of receiving at least one reactional sample of interest PROBE. This PROBE sample is subjected to an amplification treatment comprising a succession of applications of an amplification reaction by means of an amplification unit U.AMP. The amplification unit U.AMP may therefore be equipped with heating means (not represented) for the purpose of applying to the PROBE reactional sample, and more specifically to at least one target sequence present in the PROBE reactional sample, an amplification reaction (such as real time PCR) during which the reaction kinetics are monitored. For this purpose, the apparatus AP comprises a measuring unit U.MES for measuring a physical quantity representative of the evolution of at least one label present in the reactional sample of interest. The label(s) is (are) respectively specific for the target sequence(s). This physical quantity is preferably fluorescence. Furthermore, the apparatus is equipped with a processing unit U-TRT which is organized so that it can calculate the parameter F₀ (according to the method of document EP1700145) and estimate the initial population size of nucleic acids of the sample of interest. The processing unit U-TRT may be a computer. The processing unit U-TRT is equipped with a memory capable of storing computer data (permanent, Random Access Memory or RAM-type memory). The computer data may in particular include the instructions for implementing the quantitation method of the invention. Optionally, the processing unit U-TRT may be equipped with data transfer means capable of receiving a storage support (in particular: disk, CD-ROM, USB key). The processing unit U-TRT can therefore undergo updates. The application of the amplification unit U.AMP and of the measuring unit U.MES to the PROBE reactional sample of interest received on the carrier element, and also the calling up of the processing unit U-TRT, is carried out by a controller CNTR. The function of the controller CNTR can be computer-controlled or operator-controlled.

The invention is also directed towards a data storage support capable of cooperating with the processing unit U-TRT and comprising instructions for implementing the quantitation method of the invention.

The invention is described above with reference to real time PCR. It is not limited to this technique and could be applied to other nucleic acid amplification means. It could, for example, be applied to LCR. More specifically, any amplification technique will be used provided that it is possible to monitor the variation in yield of the reaction corresponding to this amplification.

1. Method for measuring the amount of nucleic acids of a target sequence in a sample of interest, comprising the steps of:
a. subjecting the sample of interest to an amplification treatment, with a batch of reagents comprising at least one nucleic acid(s) label specific for said target sequence, said amplification treatment comprising successive amplification cycles (i; from 1 to n),
b. measuring a physical quantity (Fᵢ) representative of the evolution of the label for at least a part of the amplification cycles,
c. expressing a parameter (F₀) representative of the physical quantity of the nucleic acid(s) label before any amplification cycle, using the measurements carried out in step b.,
d. estimating the initial population size (N₀^{sam}) of nucleic acid(s) of the target sequence using a conversion law, comprising contextual parameters, and applied to said parameter (F₀) expressed in step c.,
characterized in that:
in step d., said contextual parameters are pre-recorded reference parameters, these parameters being substantially independent of at least a part of the experimental conditions.

2. Method according to claim 1, characterized in that the reference parameters are determined in advance based on the behaviour of at least one reference sample of the same biological format as the sample of interest and of known initial population size N₀^{ref} of nucleic acid(s).

3. Method according to claim 1, characterized in that the conversion law in step d. is applied directly, without the need for correction by the execution in parallel of steps a., b. and c. for at least one calibrator.

4. Method according to claim 1, characterized in that step d. also comprises a correction of the estimated initial population size (N₀^{sam}) this correction being based on steps a., b. and c. being carried out in advance for at least one calibrator (EQC) of known initial population size of nucleic acid(s) and of known parameter (F₀^{EQC}) representative of the physical quantity of the nucleic acid(s) label before any amplification cycle.

5. Method according to claim 4, characterized in that the correction of the estimated initial population size (N₀^{sam}) comprises the steps of:
establishing a corrective law between:
said known parameter (F₀^{EQC}) representative of the physical quantity of the nucleic acid(s) label before any amplification cycle, firstly, and
the effectively expressed parameter (F₀^{EQCmeasured}) representative of the physical quantity of the nucleic acid(s) label before any amplification cycle, of the calibrator,
applying said corrective law to said conversion law comprising the contextual parameters of step d., in order to estimate said initial population size (N₀^{sam}) of nucleic acid(s) of the target sequence, present in the sample of interest.

6. Method according to claim 4, characterized in that said calibrator (EQC) of known initial population size is based on a biological substance able to form a positive control for the sample of interest.

7. Method according to claim 1, characterized in that the expression of the parameter (F₀) involves at least:
one parameter relating to the switch between a first phase of constant amplification yield and a second phase of non-constant amplification yield,
one parameter relating to the constant amplification yield during said first phase, and
one parameter relating to the non-constant amplification yield during said second phase.

8. Method according to claim 1, characterized in that the nucleic acids label is a fluorescent label.

9. Method according to claim 1, characterized in that the sample of interest comprises a biological specimen that may comprise a pathogenic agent.

10. Method according to claim 1, characterized in that the amplification reaction is a real time polymerase chain reaction (PCR).

11. Method according to claim 1, characterized in that said reference parameters are independent of at least one of the elements of the group of following experimental conditions: apparatus used, type of apparatus used, operator, period of validity of the batch of reagents, method of extraction of the nucleic acids of the target sequence.

12. Method according to claim 1, characterized in that the sample of interest comprises several distinct target sequences.

13. Apparatus (AP) for measuring the amount of nucleic acids of at least one target sequence in a sample of interest, comprising:
a. a carrier element (U.SUP) for carrying at least one sample (PROBE) comprising a batch of reagents and the target sequence with at least one nucleic acid(s) label specific for said target sequence,
b. an amplification unit (U.AMP) for subjecting the sample of interest to an amplification treatment comprising successive amplification cycles (i; from 1 to n),
c. a measuring unit (U.MES) for measuring a physical quantity (Fᵢ) representative of the evolution of said label for at least a part of the amplification cycles,
d. a processing unit (U.TRT) comprising a memory and organized so as:
i. to express a parameter (F₀) representative of the physical quantity of the nucleic acid(s) label prior to any of the amplification cycles, on the basis of measurements of physical quantity,
ii. to estimate the initial population size (N₀^{sam}) of nucleic acid(s) of a target sequence present in the sample of interest using a conversion law, on the basis of contextual parameters,
e. a controller (CNTR) organized so that, when a sample of interest (PROBE) is received on the carrier element (U.SUP), it applies the amplification unit (U.AMP) and the measuring unit (U.MES) to said sample (PROBE) received, and calls up the processing unit (U.TRT) with the measurements obtained by said measuring unit (U.MES),
characterized in that said contextual parameters are reference parameters which are substantially independent of at least a part of the experimental conditions and are pre-recorded in the processing unit (U.TRT).

14. Computer program product comprising instructions for implementing the method according to claim 1, and intended to be stored in the memory of the processing unit (U.TRT) in the apparatus (AP) according to claim 13.

15. Data storage support, characterized in that it comprises instructions for implementing the method according to claim 1.
